(19) United States
(12) Patent Application Publication
(10) Pub. No.: US 2008/0020040 A1
(43) Pub. Date: Jan. 24, 2008

(54) Unit dose form for administration of ibuprofen

(75) Inventors: George Tidmarsh, Portola Valley, CA (US); Barry L. Golombik, Incline Village, NV (US); Puneet Sharma, Gaithersburg, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Horizon Therapeutics, Inc., Palo Alto, CA (US)

(21) Appl. No.: 11/489,272

(22) Filed: Jul. 18, 2006

(51) Int. Cl. A61K 31/426; A61K 31/192; A61K 9/24

(52) U.S. Cl. 424/472; 514/569; 514/370

(57) ABSTRACT

An oral dosage form for administration of ibuprofen to a subject in need of ibuprofen treatment is provided, in which an oral dosage form comprising a therapeutically effective amount of ibuprofen and a therapeutically effective amount of famotidine, in separate compartments, in amounts suitable for three times per day administration.

### 1.0 FIELD OF THE INVENTION

The invention relates to pharmaceutical compositions containing ibuprofen and famotidine, and finds application in the field of medicine.

### 2.0 BACKGROUND OF THE INVENTION

Ibuprofen, a non-steroidal anti-inflammatory drug (NSAID), has been used in humans for nearly forty years. While generally regarded as safe, ibuprofen and other NSAIDs can cause gastritis, dyspepsia, and gastric and duodenal ulceration. Gastric and duodenal ulceration is a consequence of impaired mucosal integrity resulting from ibuprofen-mediated inhibition of prostaglandin synthesis. This side-effect is a particular problem for individuals who take ibuprofen for extended periods of time, such as patients suffering from rheumatoid arthritis and osteoarthritis.

The risk of developing gastric or duodenal ulceration can be reduced by cotherapy with the drug famotidine. Famotidine blocks the action of the histamine type2 (H2) receptor, leading to a reduction of acid secretion in the stomach. Reducing stomach acid with famotidine during treatment with certain nonsteroidal anti-inflammatory drugs is reported to decrease incidence of gastrointestinal ulcers (see Taha et al., 1996, “Famotidine for the prevention of gastric and duodenal ulcers caused by nonsteroidal anti-inflammatory drugs” N Engl J Med 334:1435-9, and Rostom et al., 2002, “Prevention of NSAID-induced gastrointestinal ulcers” Cochrane Database Syst Rev 4:CD002296).

Famotidine is used for treatment of heartburn, ulcers, and esophagitis at daily doses from 10 mg to 80 mg. Approved schedules of famotidine administration include 10 or 20 mg QD or BID (for treatment of heartburn), 20 mg or 40 mg QD (for healing ulcers, such as 40 mg HS for 4-8 weeks for healing duodenal ulcers), 20 mg HS (maintenance dose following healing of ulcer), 20 mg BID for 6 weeks (for treatment of gastroesophageal reflux disease), and 20 or 40 mg BID (for treatment of esophageal erosion). For treatment of Zollinger-Ellison Syndrome, a disease characterized by hypersecretion of gastric acid, doses of up to 800 mg/day have been used.

Although NSAID plus famotidine co-therapy reduces risk of developing gastric or duodenal ulceration, present therapies are not widely used. More effective methods of treatment and pharmaceutical compositions are needed. The present invention meets this and other needs.

### 3.0 BRIEF SUMMARY OF THE INVENTION

In one aspect the invention provides a solid oral dosage form having a first portion containing a therapeutically effective amount of ibuprofen (ibuprofen core), a second portion containing a therapeutically effective amount of famotidine (famotidine coat) that completely surrounds the first portion, and a barrier layer disposed between the first and second portions, where the oral dosage form contains ibuprofen and famotidine in an amount and at a ratio therapeutically effective for three times per day dosing. In one embodiment, the ibuprofen and famotidine are released into solution rapidly.

In one embodiment, the oral dosage form contains about 800 mg ibuprofen and about 26.6 mg famotidine or about 400 mg ibuprofen and about 13 mg famotidine.

In some embodiments, the oral dosage form contains ibuprofen and famotidine in a ratio in the range of 29:1 to 32:1. In some embodiments, the oral dosage form contains ibuprofen and famotidine in a ratio in the range of 30:1 to 31:1.

In some embodiments the oral dosage form of claim 1 contains about 750 mg to 850 mg ibuprofen and about 24 mg to 28 mg famotidine. In one embodiment he dosage form may contain about 375 mg to 425 mg ibuprofen and about 12 mg to 14 mg famotidine. In one embodiment the oral dosage form contains about 800 mg ibuprofen and about 26.6 mg famotidine or about 600 mg ibuprofen and about 26.6 mg famotidine or about 400 mg ibuprofen and about 13.3 mg famotidine. In one embodiment, the oral dosage form contains about 400 mg ibuprofen and about 13.3 mg famotidine. In one embodiment, the oral dosage form contains about 800 mg ibuprofen and about 26.6 mg famotidine. In one embodiment, the oral dosage form contains about 750 mg to 850 mg ibuprofen co-formulated with 24 mg to 28 mg famotidine, wherein the ibuprofen and famotidine are present in a ratio of 29:1 to 32:1

In one embodiment the oral dosage form is a tablet.

In some embodiments, the ibuprofen core of the oral dosage form comprises ibuprofen, 20-30% (w/w) lactose monohydrate; 0.1 to 2% colloidal silicon dioxide; 3-7% croscarmellose sodium; 1-3% hydroxy propyl methyl cellulose; 2-6% silicified microcrystalline cellulose and 0.1-2% magnesium stearate. In some embodiments the barrier layer is Opadry.

In one embodiment the invention provides an oral dosage form as described above, where at least 75% of the famotidine and at least 75% of the ibuprofen in the dosage form are released within 15 minutes when measured in a Type II dissolution apparatus (paddles) according to U.S. Pharmacopoeia XXIX at 37° C. in 50 mM potassium phosphate buffer, pH 7.2 at 50 rotations per minute.

In one aspect, the invention provides a solid oral dosage form comprising a first portion comprising a therapeutically effective amount of famotidine; a second portion comprising a therapeutically effective amount of ibuprofen, wherein the first portion completely surrounds the second portion; and a barrier layer disposed between the first and second portions, wherein in an aqueous environment the ibuprofen and famotidine are released into solution rapidly, and wherein the ibuprofen and famotidine are present in a range of 30:1 to 31:1.

In one aspect, the invention provides a solid oral dosage form comprising a first portion comprising a therapeutically effective amount of famotidine; a second portion comprising a therapeutically effective amount of ibuprofen, wherein the first portion completely surrounds the second portion; and a barrier layer disposed between the first and second portions, wherein in an aqueous environment the ibuprofen and famotidine are released into solution rapidly, and wherein the solid oral dosage form contains about 800 mg ibuprofen and about 26.6 mg famotidine or about 400 mg ibuprofen and about 13.3 mg famotidine.

In an aspect of the invention, a solid oral dosage form is provided having a first portion comprising a therapeutically effective amount of famotidine; a second portion comprising a therapeutically effective amount of ibuprofen, where the first portion completely surrounds the second portion; and a barrier layer disposed between the first and second portions, where in an aqueous environment the ibuprofen and famotidine are released into solution rapidly, and where the solid oral dosage form is a tablet comprising an ibuprofen-containing core portion surrounded by a famotidine-containing layer and a barrier layer interposed between the core portion and famotidine-containing layer, and wherein the core portion comprises ibuprofen, 20-30% (w/w) lactose monohydrate; 0.1 to 2% colloidal silicon dioxide; 3-7% croscarmellose sodium; 1-3% hydroxy propyl methyl cellulose; 2-6% silicified microcrystalline cellulose and 0.1-2% magnesium stearate.

In related aspects the invention provides a method of treating a patient in need of ibuprofen treatment by administering one or more a unit dose forms disclosed herein, where the administering is on a three times per day (TID) schedule. In some embodiments, TID administration of the dosage form provides better gastric protection over a 24-hour period than does TID administration of the same daily quantity of ibuprofen and two times a day (BID) administration of the same daily quantity of famotidine.

In some embodiments TID administration of a dosage form of the invention that contains 800 mg ibuprofen and 26.6 mg famotidine provides better gastric protection over a 24-hour period than separate TIED administration of 800 mg ibuprofen and BID administration of 40 mg famotidine. In preferred embodiments, the subject's intragastric pH is greater than 3.5 for at least 18 hours of a 24 hour dosing cycle, or is greater than 3.5 for at least 20 hours of a 24 hour dosing cycle during a course of treatment with said oral dosage form.

In some embodiments the patient is be a person at elevated risk for developing an NSAID-induced ulcer. The patient may be a person in need of ibuprofen treatment for a chronic condition (e.g., rheumatoid arthritis, osteoarthritis or chronic pain). The patient may be a person in need of ibuprofen treatment for acute pain, dysmenorrhea or acute inflammation.

### 4.0 BRIEF DESCRIPTION OF THE FIGURES

FIG. 1 shows the predicted effect on intragastric pH of administration of 26.6 mg famotidine TID. FIG. 1A (upper panel) shows the predicted intragastric pH during TID dosing of famotidine (80 mg/day). FIG. 1B (lower panel) shows the predicted plasma famotidine concentration during TID dosing of famotidine (80 mg/day).

FIG. 2 shows the predicted effect on intragastric pH of administration of 40 mg famotidine BID. FIG. 2A (upper panel) shows the predicted intragastric pH during BID dosing of famotidine (80 mg/day). FIG. 2B (lower panel) shows the predicted plasma famotidine concentration during BID dosing of famotidine (80 mg/day).

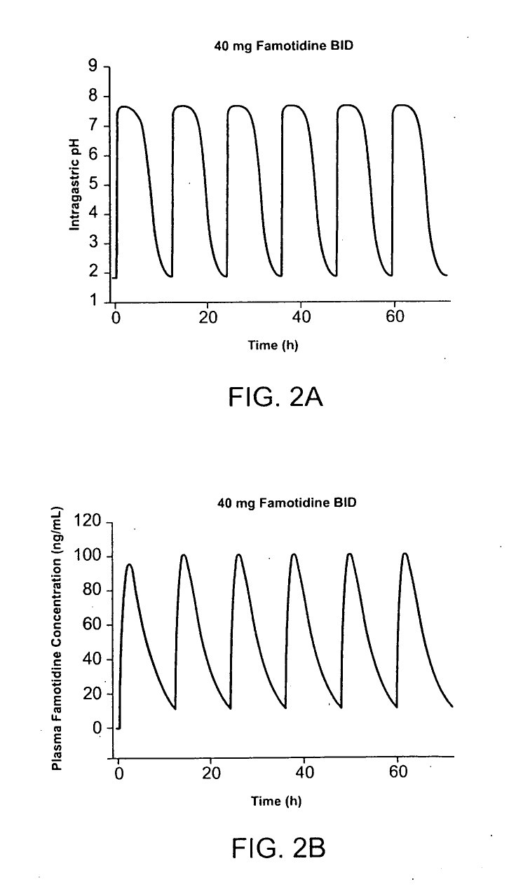

### DETAILED DESCRIPTION

### 5.0 DEFINITIONS

5.1 “Famotidine” is 3-[2-(diaminomethyleneamino)thiazol-4-ylmethylthio]-N-sulfamoylpropionamidine, including the polymorphic forms designated Form A and Form B (see, e.g. U.S. Pat. Nos. 5,128,477 and 5,120,850) and their mixtures, as well as pharmaceutically acceptable salts thereof. Famotidine can be prepared using art-known methods, such as the method described in U.S. Pat. No. 4,283,408. Famotidine properties have been described in the medical literature (see, e.g., Echizen et al., 1991, Clin Pharmacokinet. 21:178-94).

5.2 “Ibuprofen” is 2-(p-isobutylphenyl) propionic acid (C₁₃H₁₈O₂), including various crystal forms and pharmaceutically acceptable salts. Two enantiomers of ibuprofen exist. As used herein in the context of solid formulations of the invention, “ibuprofen” refers to a racemic mixture or either enantiomer (with a mixture enriched in the S-enantiomer, or a composition substantially free of the R-enantiomer preferred). Ibuprofen is available commercially and, for example, ibuprofen preparations with mean particle sizes of 25, 38, 50, or 90 microns can be obtained from BASF Aktiengesellschaft (Ludwigshafen, Germany). In one embodiment of the invention, a coated ibuprofen product, such as those described in U.S. Pat. No. 6,251,945 is used. One useful Ibuprofen product is available from BASF under the trade name Ibuprofen DC 85™ Ibuprofen's properties have been described in the medical literature (see, e.g., Davies, 1998, “Clinical pharmacokinetics of ibuprofen. The first 30 years” Clin Pharmacokinet 34:101-54).

5.3 An “API” is an active pharmaceutical ingredient. As used herein, “API” refers to ibuprofen and/or famotidine.

5.4 A “therapeutically effective amount” of ibuprofen is an amount of ibuprofen or its pharmaceutically acceptable salt which eliminates, alleviates, or provides relief of the symptoms for which it is administered.

5.5 A “therapeutically effective amount” of famotidine is an amount of famotidine or its pharmaceutically acceptable salt which suppresses gastric acid secretion.

5.6 The terms “solid oral dosage form,” “oral dosage form,” “unit dose form,” “dosage form for oral administration,” and “oral dosage unit” are used interchangably and refer to a pharmaceutical composition in the form of a tablet, capsule, caplet, gelcap, geltab and the like.

5.7 An “excipient,” as used herein, is any component of an oral dosage form that is not an API. Excipients include binders, lubricants, diluents, disintegrants, coatings, barrier layer components, glidants, and other components. Excipients are known in the art (see HANDBOOK OF PHARMACEUTICAL EXCIPIENTS, FIFTH EDITION, edited by Rowe et al., McGraw Hill). Some excipients serve multiple functions or are so-called high functionality excipients. For example, talc may act as a lubricant, and an anti-adherent, and a glidant. See Pifferi et al., 2005, “Quality and functionality of excipients” Farmaco. 54:1-14; and Zeleznik and Renak, Business Briefing: Pharmagenerics 2004.

5.8 A “binder” is an excipient that imparts cohesive qualities to components of a pharmaceutical composition. Commonly used binders include, for example, starch; sugars, such as, sucrose, glucose, dextrose, and lactose; cellulose derivatives such as powdered cellulose, microcrystalline cellulose, silicified microcrystalline cellulose (SMCC), hydroxypropylcellulose, low-substituted hydroxypropylcellulose, hypromellose (hydroxypropylmethylcellulose); and mixtures of these and similar ingredients.

5.9 A “lubricant” is an excipient added to reduce sticking by a solid formulation to the equipment used for production of a unit does form, such as, for example, the punches of a tablet press. Examples of lubricants include magnesium stearate and calcium stearate. Other lubricants include, but are not limited to, aluminum-stearate, PEG 4000-8000, talc, sodium benzoate, glyceryl mono fatty acid (e.g. glyceryl monostearate from Danisco, UK), glyceryl dibehenate (e.g. CompritolATO888™ Gattefosse France), glyceryl palmito-stearic ester (e.g. Precirol™, Gattefosse France), polyoxyethylene glycol (PEG, BASF), hydrogenated cotton seed oil or castor seed oil (Cutina H R, Henkel) and others.

5.10 A “diluent” is an excipient added to a pharmaceutical composition to increase bulk weight of the material to be formulated, e.g. tabletted, in order to achieve the desired weight.

5.11 The term “disintegrant” refers to excipients included in a pharmaceutical composition in order to ensure that the composition has an acceptable disintegration rate in an environment of use. Examples of disintegrants include starch derivatives (e.g., sodium carboxymethyl starch and pregelatinized corn starch such as starch 1500 from Colorcon) and salts of carboxymethylcellulose (e.g., sodium carboxymethylcellulose), crospovidone (cross-linked PVP polyvinylpyrrolidinone (PVP), e.g., Polyplasdone™ from ISP or Kollidon™ from BASF).

5.12 The term “glidant” is used to refer to excipients included in a pharmaceutical composition to keep the component powder flowing as the tablet is being made, preventing formation of lumps. Nonlimiting examples of glidants are colloidal silicon dioxides such as CAB-O-SIL™ (Cabot Corp.), SYLOID™, (W.R. Grace & Co.), AEROSIL™ (Degussa) talc, and corn starch.

5.13 The term “nonionic surfactant” refers to, for example and not limitation, sucrose esters; partial fatty acid esters of polyhydroxyethylenesorbitan, such as polyethylene glycol(20) sorbitan monolaurate, monopalmitate, monostearate and monooleate; polyethylene glycol(20) sorbitan tristearate and trioleate); polyethylene glycol(4) sorbitan monolaurate and monostearate; polyethylene glycol(5) sorbitan monooleate; polyhydroxyethylene fatty alcohol ethers such as polyoxyethylene cetyl stearyl ether or corresponding lauryl ethers; polyhydroxyethylene fatty acid esters; ethylene oxide/propylene oxide block copolymers; sugar ethers and sugar esters; phospholipids and their derivatives; and ethoxylated triglycerides such as the derivatives of castor oil. Examples include Cremophor™ RH 40; Cremophor™ RH 60, Tween™ 80.

5.14 The term “over-coating,” “over-coating layer,” or “over-coat” refer to the outer most coating or coatings of a unit dose form such as a tablet or caplet, which may be added to improve appearance, taste, swallowability, or other characteristics of the table or caplet. The over coating layer does not contain an API. Suitable over-coatings are soluble in, or rapidly disintegrate in water, and, for purposes of this invention, are not enteric coatings. An exemplary over-coating material is Opadry II available from Colorcon, Inc., Westpoint Pa.

5.15 “QD”, “BID”, “TID”, “QID”, and “HS” have their usual meanings of, respectively, administration of medicine once per day, twice per day, three times per day, four times per day or at bedtime. Administration three times per day means that at least 6 hours, preferably at least 7 hours, and more preferably about 8 hours elapse between administrations. Administration three times per day can mean administration about every 8 hours (e.g., 7 a.m., 3 p.m. and 11 p.m.). In some cases in which quantitative measurements are made, “TID administration” can mean administration every 8±0.25 hours.

5.16 As used herein, the term “daily quantity” refers to the quantity of an API (ibuprofen or famotidine) administered over a 24-hour period under a specific dosing regimen.

5.17 The term “barrier layer” refers a layer in the unit dosage form that does not contain an API and which is interposed between the ibuprofen-containing compartment (e.g., an ibuprofen core) and the famotidine-containing compartment (e.g., famotidine-containing coating). A barrier layer of the invention should be a water-soluble, pH independent film that promotes immediate disintegration for rapid release of ibuprofen. Materials for making barrier layers are well known in the art and include, for example and not limitation, materials are described in U.S. Pat. No. 4,543,370 (Colorcon), incorporated herein by reference. In one embodiment the barrier layer comprises a non-toxic edible polymer, edible pigment particles, an edible polymer plasticizer, and a surfactant. A preferred material, “Opadry II” is available from Colorcon (West Point Pa. USA) and comprises HPMC, titanium dioxide, plasticizer and other components.

5.18 A “subject in need of ibuprofen treatment” is an individual who receives therapeutic benefit from administration of ibuprofen. Ibuprofen is indicated for treatment of mild to moderate pain, dysmenorrhea, inflammation, and arthritis. In one embodiment, the subject in need of ibuprofen treatment is under treatment for a chronic condition. For example and without limitation, a subject in need of ibuprofen treatment may be an individual with rheumatoid arthritis, an individual with osteoarthritis, an individual suffering from chronic pain (e.g., chronic low back pain, chronic regional pain syndrome, chronic soft tissue pain), or an individual suffering from a chronic inflammatory condition. In general, a subject under treatment for a chronic condition requires ibuprofen treatment for an extended period, such as at least one month, at least four months, at least six months, or at least one year. In another embodiment, the subject in need of ibuprofen treatment is under treatment for a condition that is not chronic, such as acute pain, dysmenorrhea or acute inflammation. Preferably the patient in need of ibuprofen treatment does not suffer from a condition characterized by hypersecretion of gastric acid (e.g., Zollinger-Ellison Syndrome). Preferably the patient does not suffer from Barrett's ulceration or active severe oesophagitis. In certain embodiments the subject does not have gastroesophageal reflux disease (GERD). In certain embodiments the subject is not in need of treatment for an ulcer. In certain embodiments the subject does not suffer from dyspepsia. In certain embodiments the subject is at elevated risk of developing an NSAID-induced ulcer.

5.19 An “ibuprofen responsive condition” is a condition for which symptoms are reduced by administration of ibuprofen, such as mild to moderate pain, dysmenorrhea, inflammation, arthritis (e.g., rheumatoid arthritis and osteoarthritis), chronic pain, chronic inflammatory condition, chronic pain, acute pain and acute inflammation.

5.20 A “subject in need of famotidine treatment” is an individual who receives therapeutic benefit from administration of famotidine. In one embodiment, the subject in need of famotidine treatment requires treatment for non-ulcerative dyspepsia. In one embodiment, the subject in need of famotidine treatment requires treatment for gastroesophageal reflux disease (GERD) or for esophagitis due to GERD or for ulcer (duodenal or gastric). In one embodiment, the subject is not under NSAID therapy (e.g., does not take ibuprofen and/or a different NSAID for treatment of a chronic condition). In one embodiment, the subject in need of famotidine treatment requires treatment for dyspepsia but does not require treatment for ulcer, GERD or its complications. As used herein, “subject in need of famotidine treatment” specifically excludes any subject in need of treatment for hypersecretion of gastric acid (e.g., Zollinger-Ellison Syndrome). In certain embodiment, the patient does not suffer from Barrett's ulceration or active severe oesophagitis. In certain embodiments a “subject in need of famotidine treatment” does not suffer from gastroesophageal reflux disease (GERD) or esophagitis due to GERD. In certain embodiments a “subject in need of famotidine treatment” does not have an ulcer. In certain embodiments the subject does not suffer from dyspepsia.

5.21 A “famotidine responsive condition” is a condition for which symptoms are reduced by administration of famotidine, such as dyspepsia, GERD, esophagitis due to GERD, or ulcer.

5.22 A subject is “at elevated risk for developing an NSAID-induced ulcer” if the subject in more susceptible than the average individual to develop an ulcer when under treatment with an NSAID. A high odds ratio for risk of development of NSAID-associated ulcer complications is seen in individuals with a past complicated ulcer (odds ratio 13.5), individuals taking multiple NSAIDs or NSAIDs plus aspirin (odds ratio 9.0); individuals taking high doses of NSAIDs (odds ratio 7.0), individuals under anticoagulant therapy, such as low dose aspirin (odds ration 6.4), individuals with a past uncomplicated ulcer (odds ratio 6.1), and individuals older than 70 years (odds ratio 5.6) See, e.g., Gabriel et al., 1991, Ann Intern Med. 115:787; Garcia Rodriguez et al. 1994, Lancet 343:769; Silverstein et al. 1995, Ann Intern Med. 123:241; and Sorensen et al., 2000, Am J Gastroenterol. 95:2218. Subjects at increased risk for developing an NSAID-induced ulcer may have one or more of these risk factors. Subjects “at high risk for developing an NSAID-induced ulcer” are individuals older than 80 years of age and subjects with a history of NSAID-associated serious gastrointestinal complications (perforation of ulcers, gastric outlet obstruction due to ulcers, gastrointestinal bleeding).

5.23 “Admixture” refers to a pharmaceutical composition made by combining and mixing two or more drugs and one or more excipients in the same compartment of the unit dosage form.

5.24 A “compartment” in the context of a unit dosage form is a physical region of a tablet or other dosage form. Two components of a unit dosage form are in “separate compartments” when they are physically separated.

5.25 As used herein in the context of a unit dosage form, the term “enteric” has its usual meaning and refers to a medicinal preparation that passes through the stomach intact and disintegrates in the intestines. An “enteric coating” remains insoluble at gastric pH, then allows for release of the active ingredient from a coated particle or coated dosage form at pH greater than about 5.0, e.g. 5.5, 6.0, 6.5, or 7.0

5.26 As used herein, “dyspepsia” refers to upper abdominal pain or discomfort with or without symptoms of early satiety, nausea, or vomiting with no definable organic cause, as diagnosed following the Rome II criteria (Talley et al., 1999, Gut 45 (Suppl. II):1137-42), or any subsequent modification thereof. According to the Rome II criteria, a diagnosis of functional dyspepsia requires: (1) persistent or recurrent abdominal pain or discomfort centered in the upper abdomen; (2) symptom duration of at least 12 weeks, which need not be consecutive, within the preceding 12 months; (3) no evidence of organic disease (including at upper endoscopy) that is likely to explain symptoms; (4) no evidence that dyspepsia is exclusively relieved by defecation or association with the onset of a change in the stool frequency or stool form (i.e., not irritable bowel syndrome). In this context, “discomfort” is defined as an unpleasant sensation, and may include fullness, bloating, early satiety, and nausea. The definition includes, without limitation, ulcer-like, dysmotility-like, and non-specific dyspepsia. Symptoms of dyspepsia include nausea, regurgitation, vomiting, heartburn, prolonged abdominal fullness or bloating after a meal, stomach discomfort or pain, and early fullness.

5.27 A unit dose form is in an “aqueous environment” when it is in a water-based solution in vivo (e.g., in the stomach) or in vitro. One in vitro aqueous environment is 50 mM potassium phosphate buffer, pH 7.2. Another in vitro aqueous environment is 50 mM potassium phosphate buffer, pH 4.5.

5.28 All percentages are % w/w, unless specifically indicated otherwise. Unless otherwise indicated, “% weight” is per cent weight of the specified component compared to the total weight of the unit dosage (e.g., tablet). Optionally the % weight can be calculated as if the total weight of the unit dosage form is the weight of the ibuprofen portion, famotidine portion, and barrier layer, but not including the over-coating (e.g., added to mask taste, improve ease of swallowing, to improve appearance, and the like). Optionally the % weight can be calculated based on the total weight of the unit dosage form, including all coatings. “United States Pharmacopeia” and “USP” mean the United States Pharmacopeia and National Formulary 29th Revision (available from 12601 Twinbrook Parkway, Rockville, Md. 20852-1790, USA). It will be appreciated that due to round or practical limits on quantitive measurements, reference to a quantity of API or excipient in a dosage form can include some variation, such as ±10%, preferably ±5%, and more preferably ±1%. It will be appreciated, for example, that a total quantity of 80 mg famotidine can be administered in three doses of 26.6 mg famotidine per dose.

### 6.0 ORAL DOSAGE FORM FOR TID ADMINISTRATION OF IBUPROFEN AND FAMOTIDINE

The present invention relates to oral dosage forms comprising ibuprofen, famotidine, and one or more pharmaceutically acceptable excipients, which dosage forms are suited for TID administration to a patient in need of ibuprofen treatment.

When administered to avoid or mitigate the ulcerogenic effects of long-term NSAID therapy, famotidine is administered at 40 mg BID (see Taha et al., 1996, supra). However, it has now been determined using pharmacokinetic modeling (see Example 1) that, surprisingly, TID administration of famotidine provides a protective effect superior to that achieved by BID dosing. For example, TID administration of famotidine results in intragastric pH higher than 3.5 for a greater proportion of the dosing cycle than conventional BID dosing.

In addition, a human clinical study described in Example 3, below, has shown that the pharmocokinetic parameters for concurrent administration of immediate release forms of ibuprofen and famotidine were not significantly different from pharmocokinetic parameters for separate administration of the two APIs. When administered concurrently, both ibuprofen and famotidine retain immediate release characteristics of rapid absorption and rapid: attainment of the maximum plasma concentration (Tₘₐₓ).

These data indicate that a treatment paradigm in which ibuprofen and famotidine are administered as a unit dose form on a TID (three times per day) schedule will deliver ibuprofen that is bioequivalent to that of conventional TID dosing of ibuprofen, while providing significant and superior protection from ibuprofen-related side effects such as increased likelihood ulcer development and dyspepsia. Administration of ibuprofen-famotidine TID will provide superior protection, as measured by gastric pH, compared to cotherapy with famotidine QD and ibuprofen TID.

It has also been discovered, as described in the examples below, that ibuprofen and famotidine are pharmaceutically incompatible when formulated as an admixture and stored under “forced degradation” conditions. See Examples 4 and 5. Forced degradation conditions refer to conditions of elevated temperature, or elevated temperature and humidity, intended to accelerate the process of chemical degradation. Forced degradation conditions for a period of time are used to predict the effect of storage under more benign conditions (e.g., room temperature) for a longer period of time. The present invention overcomes this incompatibility by formulating the ibuprofen and famotidine in separate compartments of the dosage form.

### 7.0 IBUPROFEN-FAMOTIDINE ORAL DOSAGE FORMS: API CONTENT

The dosage forms of the invention comprise ibuprofen and famotidine in amounts sufficient to provide therapeutic efficacy when administered three times per day. At each administration time the therapeutically appropriate amount of drug can be administered as a single unit dosage form (e.g., tablet) or as multiple unit doses such as a split dose (i.e., the drug is administered as two tablets taken together). For example, TID administration of 800 mg ibuprofen and 26.6 mg famotidine can be in the form of a single unit dosage form containing 800 mg ibuprofen and about 26.6 mg famotidine, two unit dosage forms containing 400 mg ibuprofen and about 13.3 mg famotidine, or even four unit dosage forms containing 200 mg ibuprofen and about 7 mg famotidine. Preferably, a therapeutically effective dose is administered as one or two tablets.

In general, the unit dosage forms of the invention comprise ibuprofen in an amount of about 50-1000 mg and more often about 200-800 mg. A therapeutically effective dose for headache or mild pain may be 200 mg or 400 mg TID. A therapeutically effective dose for arthritis is usually 800 mg TID. In preferred embodiments the unit dosage forms of the invention contain about 200, about 400, about 600 or about 800 mg ibuprofen.

For many applications the quantity of ibuprofen in the unit dose form is about 800 mg (e.g., in the range 750 mg to 850 mg) which allows administration of 2400 mg/day with TID administration of one tablet, or the quantity of ibuprofen is about 400 mg (e.g., in the range 375 mg to 425 mg) which allows administration of 2400 mg/day with TID administration of two tablets. In some embodiments the quantity of ibuprofen is about 600 mg (e.g., in the range 575 mg to 625 mg). In some embodiments the quantity of ibuprofen is about 200 mg (e.g., in the range 175 mg to 225 mg)

In general, the unit dosage forms of the invention comprise famotidine in an amount of about in the range 7 mg to 30 mg, more often in the range of 12 mg to 28 mg. For many applications the quantity of famotidine in the unit dose form is about 26.6 mg (e.g., in the range 24 mg to 28 mg) which allows administration of 80 mg/day with TID administration of one tablet, or the quantity of famotidine is about 13 mg (e.g., in the range 12 mg to 14 mg) which allows administration of 80 mg/day with TID administration of two tablets.

In one preferred embodiment, the oral unit dosage forms are formulated to deliver a daily dose of about 2400 mg ibuprofen and about 80 mg famotidine with three times per day administration. For many applications the quantity of ibuprofen is about 800 mg (e.g., in the range 750 mg to 850 mg) and the quantity of famotidine is about 26.6 mg (e.g., in the range 24 mg to 28 mg). This allows administration of 2400 mg/day ibuprofen and 80 mg/day famotidine with TID administration of one tablet. In a related embodiment, the quantity of ibuprofen is about 400 mg (e.g., in the range 375 mg to 425 mg) and the quantity of famotidine is about 13 mg (e.g., in the range 12 mg to 14 mg). This allows administration of 2400 mg/day ibuprofen and 80 mg/day famotidine with TID administration of two tablets. In a related embodiment, the quantity of ibuprofen is about 200 mg (e.g., in the range 175 mg to 225 mg) and the quantity of famotidine is about 6.6 mg (e.g., in the range 6 mg to 7 mg). In some embodiments the quantity of ibuprofen is about 600 mg (e.g., in the range 575 mg to 625 mg) and the quantity of famotidine is about 26.6 mg (e.g., in the range 24 mg to 28 mg).

In one embodiment, the oral unit dosage forms are formulated to deliver a daily dose of about 1800 mg ibuprofen and about 80 mg famotidine with three times per day administration. For many applications the quantity of ibuprofen is about 600 mg (e.g., in the range 550 mg to 650 mg) and the quantity of famotidine is about 26.6 mg (e.g., in the range 24 mg to 28 mg). This allows administration of 1800 mg/day ibuprofen and 80 mg/day famotidine with TID administration of one tablet. In a related embodiment, the quantity of ibuprofen is about 300 mg (e.g., in the range 275 mg to 325 mg) and the quantity of famotidine is about 13 mg (e.g., in the range 12 mg to 14 mg). This allows administration of 1800 mg/day ibuprofen and 80 mg/day famotidine with TID administration of two tablets.

In other embodiments more or less API may be administered. For example, in some cases the daily dose of ibuprofen is greater than 2400 mg (e.g., 3200 mg). This amount can easily be administered as, for example, three or six tablets per day, particularly using an ibuprofen formulation that can be tabletted with little excipient (e.g., BASF Ibuprofen DC 85®). If a formulation that contains only the active S-enantiomer of ibuprofen is used, a smaller quantity may sometimes be administered, such as about half as much as described hereinabove.

In certain embodiments the ratio of ibuprofen to famotidine in the dosage forms of the invention is in the range of 15:1 to 40:1, more often 20:1 to 40:1 and even more often 25:1 to 35:1. In some embodiments the ratio of ibuprofen to famotidine in the dosage forms of the invention is in the range of 29:1 to 32:1, such as 30:1 to 31:1. In one embodiment the ratio of ibuprofen to famotidine is about 30:1. Exemplary amounts of ibuprofen and famotidine include 800±10% mg ibuprofen and 26.6±10% mg famotidine; 600±10% mg ibuprofen and 19.95±10% mg famotidine; 400±10% mg ibuprofen and 13.3±10% mg famotidine; and 200±10% mg ibuprofen and 6.65±10% mg famotidine.

In certain embodiments the ratio of ibuprofen to famotidine in the dosage forms of the invention is in the range of range of 20:1 to 25:1, such as 22:1 to 23:1. In one embodiment the ratio of ibuprofen to famotidine is about 22.5:1. Exemplary amounts of ibuprofen and famotidine include 600±10% mg ibuprofen and 26.6±10% mg famotidine.

In a preferred embodiment, the oral dosage form does not contain a pharmaceutically active compound (i.e., drug compound) other than ibuprofen and famotidine. In particular embodiments the oral dosage form does not contain any NSAID other than ibuprofen and/or does not contain any H2-receptor antagonist other than famotidine. In certain embodiments the amount ot famotidine is other than 5 mg, other than 10 mg, other than 20 mg or other than 40 mg per dosage form.

### 8.0 IBUPROFEN-FAMOTIDINE ORAL DOSAGE FORMS: DISSOLUTION PROPERTIES (RAPID RELEASE OF FAMOTIDINE AND IBUPROFEN)

Oral dosage forms of the invention are formulated so that release of both APIs occurs (or begins to occur) at about the same time. That is, the dosage form is not designed so that one of the APIs is released significantly later than the other API. For example, the barrier layer (described below), if present, is not designed to significantly delay release of the API contained within it. Combinations of excipients (which may include one or more of a binder, a lubricant, a diluent, a disintegrant, a glidant and oher componants) are selected which do substantially retard release of an API. See e.g., HANDBOOK OF PHARMACEUTICAL MANUFACTURING FORMULATIONs, 2004, Ed. Sarfaraz K Niazi, CRC Press; HANDBOOK OF PHARMACEUTICAL ADDITIVES, SECOND EDITION, 2002, compiled by Michael and Irene Ash, Synapse Books; and REMINGTON SCIENCE AND PRACTICE OF PHARMACY, 2005, David B. Troy (Editor), Lippincott Williams & Wilkins.

A. Release at Neutral pH

In an embodiment the unit dosage form is formulated so that famotidine and ibuprofen are released rapidly under neutral pH conditions (e.g., an aqueous solution at about pH 6.8 to about pH 7.4). In this context “rapidly” means that both APIs are significantly released released into solution within 20 minutes under in vitro assay conditions. In some embodiments both APIs are significantly released released into solution within 15 minutes under in vitro assay conditions. In this context, “significantly released” means that at least about 60% of the weight of the API in the unit dosage form is dissolved, preferably at least about 75%, more preferably at least about 80%, often at least 90%, and sometimes at least about 95%.

Dissolution rates may be determined using the known methods. Generally an in vitro dissolution assay is carried out by placing the famotidine-ibuprofen unit dosage form(s) (e.g., tablet(s)) in a known volume of dissolution medium in a container with a suitable stirring device. Samples of the medium are withdrawn at various times and analyzed for dissolved active substance to determine the rate of dissolution. Dissolution may be measured as described for ibuprofen in the USP or, alternatively, as described for famotidine in the USP. One approach is illustrated in Example 6. Briefly, the unit dose form (e.g., tablet) is placed in a vessel of a United States Pharmacopeia dissolution apparatus II (Paddles) containing 900 ml dissolution medium at 37° C. The paddle speed is 50 RPM. Independent measurements are made for at least three (3) tablets. In one suitable in vitro assay, dissolution is measured using a neutral dissolution medium such as 50 mM potassium phosphate buffer, pH 7.2 (“neutral conditions”) generally as described in Example 6, below.

B. Substantial Release of Famotidine and Ibuprofen Under Low pH Conditions

In an embodiment the unit dosage form is formulated so that famotidine and ibuprofen are both released rapidly under low pH conditions. Release under low pH conditions is measured using the assay described above and in Example 5, but using 50 mM potassium phosphate buffer, pH 4.5 as a dissolution medium. In some embodiments, a substantial amount of both APIs is released into solution within 60 minutes under low pH assay conditions. In some embodiments, a substantial amount of both APIs is released into solution within 40 minutes under low pH assay conditions. As used in this context, the APIs are released rapidly at low pH when, a substantial amount of both APIs is released into solution within 20 minutes under low pH assay conditions. In some embodiments, a substantial amount of both APIs is released into solution within 10 minutes under low pH assay conditions. In this context, a “substantial amount” means at least 15%, preferably at least 20%, and most preferably at least 25% of ibuprofen is dissolved and at least 80%, preferably at least 85%, and most preferably at least 90% of famotidine is dissolved.

### 9.0 IBUPROFEN-FAMOTIDINE ORAL DOSAGE FORMS: PROTECTIVE PROPERTIES

When administered to a subject in need of treatment with ibuprofen, the oral dosage units of the invention have properties superior to conventional medicaments. As illustrated in Example 1, TID administration to a subject of famotidine results in an intragastric pH that is elevated relative to the intragastric pH resulting from conventional BID administration of famotidine, resulting in better gastric protection. As used herein administration of a pharmaceutical composition or compositions “provides better gastric protection” compared to administration of a reference composition or compositions when administration of the pharmaceutical composition maintains stomach pH at a more basic level. It has now been discovered that TID administration of famotidine provides better gastric protection than conventional BID dosing of the same daily dose of drug.

One measure of gastric protection is the fraction of a 24-hour dosing cycle during which amount of time pH is maintained above a designated value (e.g., pH 3.0, sometimes pH 3.5, sometimes pH 4.0, and sometimes pH 4.5). For example, better gastric protection can be characterized as pH above the designated value for more time (e.g., 20 hours in a 24 hour period vs. 15 hours in a 24 hour period) than administration of the reference composition(s). In one embodiment, TID administration of famotidine (or, alternatively a unit dosage form of the invention containing famotidine and ibuprofen) will maintain a gastric pH of 3.5 or greater for at least 16, at least 17, at least 18, at least 19, at least 20, at least 21, at least 22, or at least 23 hours of a 24 hour dosing cycle. In one embodiment, TID administration of famotidine (or, alternatively a unit dosage form of the invention containing famotidine and ibuprofen) will maintain a gastric pH of 3.0 or greater for at least 16, at least 17, at least 18, at least 19, at least 20, at least 21, at least 22, or at least 23 hours of a 24 hour dosing cycle. In one embodiment, TID administration of famotidine (or, alternatively a unit dosage form of the invention containing famotidine and ibuprofen) will maintain a gastric pH of 3.5 or greater for at least 16, at least 17, at least 18, at least 19, at least 20, at least 21, at least 22, or at least 23 hours of a 24 hour dosing cycle. In one embodiment, TID administration of famotidine (or, alternatively a unit dosage form of the invention containing famotidine and ibuprofen) will maintain a gastric pH of 4.0 or greater for at least 16, at least 17, at least 18, at least 19, at least 20, at least 21, at least 22, or at least 23 hours of a 24 hour dosing cycle. TID administration of famotidine (or, alternatively a unit dosage form of the invention containing famotidine and ibuprofen) will maintain gastric pH of 4.5 or greater for at least 16, at least 17, at least 18, at least 19, at least 20, at least 21, at least 22, or at least 23 hours of a 24 hour dosing cycle. In one embodiment of the present invention, TID administration of famotidine (or, alternatively TID administration a unit dosage form of the invention containing famotidine and ibuprofen) results in a gastric pH above a specified value (e.g., at least 3.0, at least 3.5, at least 4.0 or at least 4.5) for more hours in a 24-hour dosing cycle that than BID administration of the same daily dose of famotidine (or, alternatively a BID administration of the same daily dose of famotidine and TID administration of the same daily dose of ibuprofen) where the difference in hours is at least 1, at least 2, at least 3, at least 4, or at least 5.

Another measure of gastric protection is the minimum sustained gastric pH during a 24-hour dosing cycle. “Sustained pH” refers to a gastric pH (or pH range) sustained for at least 10 minutes. Better gastric protection can be characterized as a higher minimum sustained pH when measured over a 24-hour dosing period. In one embodiment of the present invention, TID administration of famotidine (or, alternatively a unit dosage form of the invention containing famotidine and ibuprofen) results in a minimum sustained pH of at least 2.0, preferably at least 2.3, more preferably at least 2.5, and sometimes at least 3.0. In one embodiment of the present invention, TID administration of famotidine (or, alternatively TID administration a unit dosage form of the invention containing famotidine and ibuprofen) results in a minimum sustained pH that is higher than BID administration of the same daily dose of famotidine (or, alternatively a BID administration of the same daily dose of famotidine and TID administration of the same daily dose of ibuprofen) where the difference in pH is at least 0.2, at least 0.4, at least 0.5, at least 0.6, or at least 0.7 pH units.

Another measure of gastric protection is the average or median gastric pH during a 24-hour dosing cycle. Better gastric protection can be characterized as a higher average or median gastric pH over a 24-hour dosing period. In one embodiment of the present invention, TID administration of famotidine (or, alternatively a unit dosage form of the invention containing famotidine and ibuprofen) results in an average or median gastric pH of at least 6.0, preferably at least 6.1, more preferably at least 6.2, even more preferably at least 6.3 and sometimes at least 6.4. In one embodiment of the present invention, TID administration of famotidine (or, alternatively TID administration a unit dosage form of the invention containing famotidine and ibuprofen) results in an average or median gastric pH that is higher than BID administration of the same daily dose of famotidine (or, alternatively a BID administration of the same daily dose of famotidine and TID administration of the same daily dose of ibuprofen) where the difference in pH is at least 0.2, at least 0.3, at least 0.4, at least 0.6, at least 0.7 or at least 0.8 pH units.

For illustration, TID administration of a unit dosage form containing 800 mg ibuprofen and 26.6 mg famotidine would provide superior gastric protection than does TID administration of a unit dosage form containing 800 mg ibuprofen and BID administration of a unit dosage form containing 40 mg famotidine.

Intragastric pH can be determined by art-known methods using, for example, a nasogastric pH probe. One useful probe is the Digitrapper:T pH 400 ambulatory pH recorder from Medtronic Functional Diagnostics (Shoreview, Minn.). Measurements can be made after the subject has received the appropriate dosage regimen for 3 days, which allows steady state levels of drug to be achieved.

### 10.0 UNIT DOSE FORM

In a preferred embodiment, the invention provides a solid oral dosage form with a first portion comprising a therapeutically effective amount of ibuprofen and a second portion comprising a therapeutically effective amount of famotidine, where the famotidine portion completely surrounds the ibuprofen portion; and a barrier layer disposed between the two portions.

The API content of the unit dose forms is selected so that TID administration delivers a therapeutically effective dose of ibuprofen and a therapeutically effective dose of famotidine. Preferably the oral dosage form comprises ibuprofen and famotidine in the amounts and ratios described hereinabove.

According to the invention, famotidine and ibuprofen are released rapidly, as described above. It will be recognized, therefore, that in this aspect of the invention neither the dosage nor the APIs individually are enteric coated or formulated for sustained or delayed release. The tablets are formulated so that they disintegrate in the stomach after they are swallowed and do not dissolve in the mouth or throat during the normal process of oral administration. Other properties of the oral dosage forms of the invention will be apparent to the reader.

In this version, the oral dosage form comprises an ibuprofen core (“core”), a surrounding layer containing famotidine (“famotidine layer”) and a barrier layer interposed between the core and famotidine layer. Usually the famotidine coat entirely surrounds the ibuprofen core. Optionally the tablet is coated by one or more over-coating layers, for example, to improve appearance, taste, swallowability, or for other reasons. Methods for formulation and manufacture of pharmaceutical unit dose forms are known in the art, see, e.g., HANDBOOK OF PHARMACEUTICAL MANUFACTURING FORMULATIONs, 2004, Ed. Sarfaraz K Niazi, CRC Press; HANDBOOK OF PHARMACEUTICAL ADDITIVES, SECOND EDITION, 2002, compiled by Michael and Irene Ash, Synapse Books; and REMINGTON SCIENCE AND PRACTICE OF PHARMACY, 2005, David B. Troy (Editor), Lippincott Williams & Wilkins. One of ordinary skill in the art guided by this disclosure will be able to make a variety of suitable oral unit dose forms.

10.1 Ibuprofen-Containing Core

The ibuprofen core may vary in shape and may be, for example, round, ovoid, oblong, cylindrical (e.g., disk shaped) or any other suitable geometric shape, for example rectilinear. Preferably the tablet has a disk or ovoid shape is shaped like a flattened disk, ovoid or torpedo. In one embodiment, the core does not have an internal hole extending all or part-way through the pill. For example, the core is not shaped like a cup or donut.

The tablet of the invention comprises a therapeutically effective amount of ibuprofen API e.g., in the amounts described above (e.g., about 200, about 400, about 600 or about 800 mg). In addition to ibuprofen the core may contain excipients such as one or more disintegrants, binders, glidants, or lubricants. For example, the core may contain lactose (e.g., lactose monohydrate); colloidal silicon dioxide; sodium croscarmellose; hydroxy propyl methyl cellulose; silicified microcrystalline cellulose and/or magnesium stearate. In one embodiment ibuprofen core comprises is ibuprofen, 20-30% (w/w) lactose monohydrate; 0.1 to 2% colloidal silicon dioxide; 3-7% crosscarmellose sodium; 1-3% hydroxy propyl methyl cellulose; 2-6% silicified microcrystalline cellulose (Prosolv SMCC 90) and 0.1-2% magnesium stearate. In some embodiments, the core does not contain a lubricant.

In one embodiment, the core comprises Ibuprofen DC 85 (BASF) which comprises 85% API. Exemplary formulations using Ibuprofen DC 85, for illustration and not limitation, include:

1) Ibuprofen DC 85 88.24% (w/w); microcrystalline cellulose 7.76%; crosslinked sodium carboxymethylcellulose 3.00%; silica 0.05%; and magnesium stearate 0.50%;

2) Ibuprofen DC 85 88.24% (w/w); corn starch 7.76%; crosslinked sodium carboxymethylcellulose 3.00%; silica 0.05% and magnesium stearate 0.50%;

3) Ibuprofen DC 85 88.24% (w/w); lactose 7.76%; crosslinked sodium carboxymethylcellulose 3.00%; silica 0.05% and magnesium stearate 0.50%.

The core may be formed using art-known techniques including wet granulation, dry granulation, direct compression or any other pharmaceutically acceptable process. The appropriate amount of the ibuprofen formulation (i.e., the amount containing the unit dose of API) may be compression pressed into individual cores. Alternatively, the core may be formed by molding.

In one embodiment, the core portion is at least 50% ibuprofen by weight, preferably at least 60%, and more preferably at least 70%, and even more preferably at least 80% ibuprofen.

10.2 The Barrier Layer

The barrier layer may be composed of any of a variety of materials that (1) separate the core and famotidine layer and (2) rapidly disintegrate in an aqueous (e.g., gastric) environment so that the ibuprofen is rapidly released.

The barrier layer may comprise fillers, binders, disintegrants, lubricants, glidants, and the like, as known in the art. Suitable fillers for use in making the barrier layer, or a portion thereof, by compression include water-soluble compressible carbohydrates such as sugars, which include dextrose, sucrose, maltose, and lactose, sugar-alcohols, which include mannitol, sorbitol, maltitol, xylitol, starch hydrolysates, which include dextrins, and maltodextrins,

In a preferred embodiment, the ibuprofen cores are coated with Opadry II white (Colorcon Y-22-7719) according to manufacturer's instructions to a weight gain of 1.5-2.0% w/w. Other known barrier layer materials include hydroxypropyl methylcellulose phthalate, polyvinyl acetate phthalate, and cellulose acetate phthalate. In one embodiment, the formulation will contain at least one coating layer polymer and a coating solvent (preferably water) used for processing and removed by drying. The coating layer polymer may be hydroxypropyl methylcellulose, polyvinyl alcohol (PVA), ethyl cellulose, methacrylic polymers or hydroxypropyl cellulose. A plasticizer (e.g., triacetin, diethyl phthalate, tributyl sebacate or polyethylene glycol) may also be included. The coating layer may include an anti-adherent or glidant (e.g., talc, fumed silica or magnesium stearate) and colorants such as titanium dioxide, iron oxide based colorants or others.

The thickness of the barrier layer can vary over a wide range, but is generally in the range 20 to 3,000 microns, such as on the order of about 25 250 microns. Preferably the barrier layer, retards the availability of ibuprofen by less than 5 minutes, preferable less than 4 minutes and more preferably by less than 3 minutes.

The barrier layer may be formed by any method, including compression, molding, dipping, or spray coating.

10.3 The Famotidine Coat

The famotidine layer is applied over the barrier coat. The famotidine layer can be applied by compression, spray coating, or other methods. In a preferred embodiment, the famotidine layer is applied by spray coating a formulation containing famotidine and excipients such as polymers, plasticizers, and the like. In one example, famotidine is combined with Opadry II (Colorcon) and spray coated over the ibuprofen core or barrier layer.

The dosage form of the invention comprises a therapeutically effective amount of famotidine API. For many applications the quantity of famotidine is about 26.6 mg (e.g., in the range 24 mg to 28 mg) which allows administration of 80 mg/day with TID administration of one tablet, or the quantity of famotidine is about 13 mg (e.g., in the range 12 mg to 14 mg) which allows administration of 80 mg/day with TID administration of two tablets.

10.4 Over Coating Layer

In some embodiments, the tablets are coated for oral administration, to make the tablet easier to swallow, to mask taste, for cosmetic reasons, or for other reasons. Coating of tablets and caplets is well known in the art. Coating systems are typically mixtures of polymers, plasticisers, coloring agents and other excipients, which can be stirred into water or an organic solvent to produce a dispersion for the film coating of solid oral dosage forms such as tablets.

Usually a readily soluble film is used. Materials that can be used for readily soluble films include cellulose derivatives (such as hydroxypropylmethyl cellulose) or amino-alkylmethacrylate copolymers (e.g. Eudragit™E). Suitable coat layers, for illustration and not limitation, include Kollicoat® IR (a polyvinyl alcohol-polyethylene glycol graft copolymer) and Kollicoat IR White® both manufactured by BASF Aktiengesellschaft (Ludwigshafen, Germany).

The edges of the tablets may be beveled or rounded. The tablet may also be shaped as a caplet (capsule form tablet). The tablets may be scored, embossed or engraved.

### 11.0 METHOD OF MANUFACTURE OF ORAL DOSAGE FORM

It is within the ability of one of ordinary skill in the art, guided by the present disclosure and with reference to the pharmaceutical literature, to prepare and manufacture unit dosage forms of the invention.

For example, for illustration and not for limitation, in one approach an oral dosage form of Form 1 (above) uses wet granulation. A dry mix containing ibuprofen, a binder or binders (e.g., lactose monohydrate, hydroxy propyl methyl cellulose), disintegrant (e.g., crosscarmellose sodium) and glidant (e.g., colloidal silicon dioxide) is prepared. an aqueous solution containing a binder (e.g., hydroxy propyl methyl cellulose) is blended with the dry mix. the resulting wet material is milled and dried to form granules. The granules are blended with binder (e.g., silicified microcrystalline cellulose), disintegrant (e.g., crosscarmellose sodium), glidant (e.g., colloidal silicon dioxide) and lubricant (e.g., magnesium stearate). The final blend is compressed (e.g., using a DC 16 compression machine) to form the cores.

A barrier coat of Opadry II (Colorcon) is applied by spray coating according to the manufacturer's instructions. For example, one part Opadry II concentrate is added to four parts (by weight) distilled water with stirring to form a dispersion. The ibuprofen core tablets are placed in a rotating pan in a chamber where the temperature is maintained at 60-70° C. in order to control product temperature at 40-45° C. The famotidine-containing coating material is sprayed using a spray gun above the pan. (It can be expected that approximately 75% of the famotidine will coat the core, with about 25% lost during the coating process.) For example, and not for limitation, an Accela-Cota 60 inch pan equipped with four mixing baffles rotating at 5 rpm may be used. The spray apparatus may be the Five Spraying Systems ¼ JAU air gun using 2850 fluid nozzles, 134255-45 aircaps and 60 psi atomizing air. The delivery system may be a pressure pot, The delivery rate may be 110 g/min/gun.

A famotidine lay can then be applied. A polymer containing famotidine can be applied to the coated core by, for example, spray coating or compression methods known in the art. In one approach, famotidine is mixed with Opadry II (Colorcon) in an about 1:1 ratio and applied generally as described above.

An overcoating layer can then be applied. In one embodiment, after the famotidine layer is applied, the unit dose forms are coated with Opadry II (Colorcon, Inc. West Point Pa.).

### 12.0 PACKAGING

In one aspect the invention provides a container, such as a vial, containing a one-month supply of ibuprofen/famotidine tablets of the invention, wherein the number of tablets in the container is from 89-94 tablets (e.g., 89, 90, 91, 92, 93 or 94 tablets), and wherein instructions to take the medication 3× daily are affixed to the container, or packaged with the container.

Also provided is a container containing a two-month supply of ibuprofen/famotidine tablets of the invention, wherein the number of tablets in the container is 178-188 tablets, and wherein instructions to take the medication 3× daily are affixed to the container or packaged with the container.

### 13.0 METHOD OF TREATMENT

The oral dosage forms of the invention are administered (e.g. prescribed) to subjects in need of ibuprofen treatment and to treat any ibuprofen-responsive condition. In one embodiment the patient is instructed to ingest the drug tablets three times daily. In one embodiment the patient is instructed to ensure there is at least a 6-hr interval between administrations of consecutive doses.

For illustration and not limitation, the oral dosage forms of the invention may be administered to a patient in need of ibuprofen treatment for mild to moderate pain, dysmenorrhea, inflammation, and arthritis. In one embodiment, the subject in need of ibuprofen treatment is under treatment for a chronic condition. For example and without limitation, a subject in need of ibuprofen treatment may be an individual with rheumatoid arthritis, an individual with osteoarthritis, an individual suffering from chronic pain (e.g., chronic low back pain, chronic regional pain syndrome, chronic soft tissue pain), or an individual suffering from a chronic inflammatory condition.

The oral dosage forms of the invention may be administered to a patient is at elevated risk for developing an NSAID-induced ulcer. The oral dosage forms of the invention may be administered to a subject in need of ibuprofen treatment to reduce the risk that the patient will develop an ibuprofen-induced symptom or condition treatment such as ulcer or GERD.

### 14.0 EXAMPLES

### 14.1 Example 1

### Administration of Famotidine TID Provides Superior Gastric Protection Compared to Administration of Famotidine QD

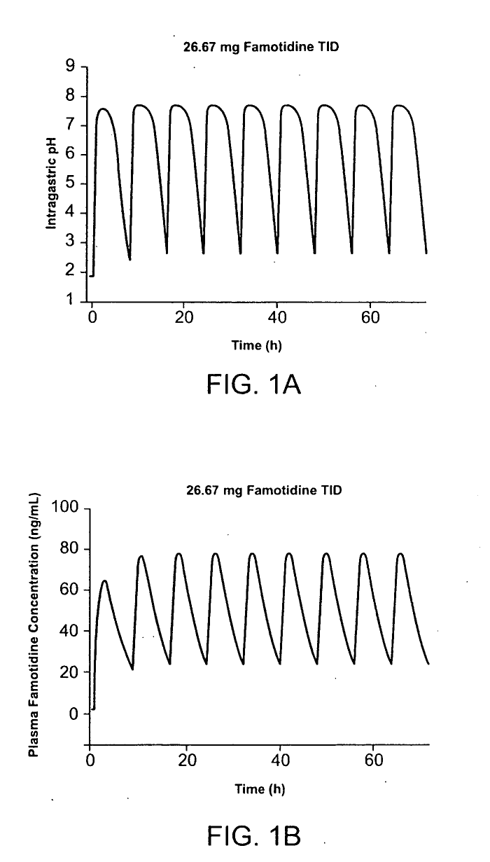

Pharmocokinetic modeling shows that TID administration of famotidine and ibuprofen according to the method of the present invention provides protection superior to that achieved by conventional cotherapy. FIG. 1A shows the predicted effect on intragastric pH of administration of 26.6 mg famotidine TID. FIG. 1B shows the predicted effect on intragastric pH of administration of 40 mg famotidine BID. Modeling shows that over a twenty-four hour interval, intragastric pH is greater than 3.5 during for several more hours per day than achieved using TID administration of famotidine compared to conventional BID dosing. In FIG. 1, administration of 80 mg/day famotidine using TID dosing is shown to maintain pH greater than 3.5 for about 21 hours per twenty-four hour interval, while the same daily dose administered BID dosing maintains pH greater than 3.5 for about 17 hours per twenty-four hour interval. The precise duration of pH elevation can be confirmed in clinical trials and may deviate somewhat from the predicted values (with the TID dosing remaining more effective than the BID dosing).

Methodology: Mean plasma concentration versus time data from a single dose bioequivalence study (www.fda.gov/cder/foi/anda/2001/75-311_Famotidine_Bioeqr.pdf, n=30) comparing 40 mg Pepcid and generic famotidine (Teva Pharm) were best fitted to a one compartment oral absorption model with a lag time using a nonlinear least-squares regression program, WinNonlin (Pharsight®). The following pharmacokinetic parameters for Pepcid were obtained:

| Parameter | Units | Estimate |
|---|---|---|
| V/F | L | 241.8 |
| kₐ | h⁻¹ | 0.8133 |
| kₑₗ | h⁻¹ | 0.2643 |
| T_{lag} | h | 0.3677 |

where V/F is the apparent volume of distribution, kₐ is the absorption rate constant, kₑₗ is the elimination rate constant and T_{lag} is the absorption lag time. The relationship between plasma concentrations of Pepcid and intragastric pH in one patient were digitized from FIG. 4 of Echizen and Ishizaki, supra, page 189. The digitized plasma concentration vs. intragastric pH were fitted using a nonlinear least-squares regression program, WinNonlin to a sigmoid Emax model using the following equation:

$$E = {E}_{o} + \frac{{E}_{max} * {C}^{γ}}{{EC}_{50}^{γ} * {C}^{γ}}$$

where E is the intragastric pH at C, Eₒ is the intragastric pH at time zero, Eₘₐₓ is the maximum intragastric pH, EC₅₀ is the Pepcid concentration at one-half of Emax, C is the plasma concentration of Pepcid and γ is the shape factor. The estimated pharmacodynamic parameters are listed below:

| Parameter | Units | Estimate |
|---|---|---|
| Eₘₐₓ | — | 7.80 |
| EC₅₀ | ng/mL | 32.6 |
| E₀ | — | 1.88 |
| γ | — | 4.80 |

### Using the pharmacokinetic parameters obtained above together with the pharmacodynamic parameters above, plasma concentrations as well as intragastric pH as a function of time were simulated for various dose regimens.

### 14.2 Example 2

### Administration of Famotidine TID Provides Superior Gastric Protection Compared to Administration of Famotidine QD

A randomized, open-label, two-period, crossover study is carried out to compare the effects on gastric pH of administration of 80 mg per day of famotidine when administered for five consecutive days in two versus three divided doses each day.

Healthy male or female subjects, age 18-45 years inclusive, are randomized to treatment to ensure that at least 12 subjects will complete study participation. Subjects are assigned randomly, in approximately a 1:1 ratio, to one of two, two-period treatment sequences as follows:
Treatment Sequence 1: 40 mg famotidine BID×5 days, followed by 26.6 mg famotidine TID×5 days.
Treatment Sequence 2: 26.6 mg famotidine TID×5 days, followed by 40 mg famotidine BID×5 days.

There is a washout of at least one week between administration of the last dose of Treatment Period 1 and administration of the first dose of Treatment Period 2.

PEPCID® (famotidine) for Oral Suspension (Merck & Co., Inc., 40 mg/5 mL) is administered with water. During treatment periods in which famotidine is to be administered TID, medication is administered at approximately 0800, 1600, and 2400 on each day of dosing. During treatment periods in which famotidine is to be administered BID, medication is administered at approximately 0800 and 2000 on each day of dosing.

Gastric pH is measured continuously, using a nasogastric pH probe, during the 24 hours following administration of the first dose of study medication on Study Day 1, and during the 24 hours following administration of the first dose of study medication on Study Day 5, during both treatment periods. Blood samples are collected prior to initiation of dosing, and prior to administration of the second dose of study medication on Study Day 1 and Study Day 5 during both treatment periods for determination of trough plasma famotidine concentrations.

The effect of each dose regimen, and the difference between the two dosing regimens, is estimated by the 95% confidence intervals for the variables (i) mean and median pH during the final 24-hour measurement period of each treatment period, and (ii) percentage of time during the final 24-hour measurement period of each treatment period in which the pH is below 4, when 80 mg doses of famotidine are administered for five consecutive days in two versus three divided doses each day. An analysis of variance (ANOVA) is performed to estimate the effects of each dose regimen and to compare the two dosing regimens for both efficacy variables.

It is expected that administration of famotidine TID provides superior protection, as measured by gastric pH, compared to therapy with famotidine BID. TID administration of famotidine maintains a gastric pH greater than pH 3.0 more than 1 hour longer per 24-hour dosing cycle than does BID administration. TID administration of famotidine results in a minimum sustained pH that is at least 0.2 pH units higher than BID administration. TID administration of famotidine results in an average gastric pH that is at least 0.2 pH units higher than BID administration.

### 14.3 Example 3

### Pharmacokinetic Drug-Drug Interaction Study of Ibuprofen and Famotidine in Healthy Male Subjects

This example demonstrates that pharmocokinetic parameters of concurrent administration of ibuprofen and famotidine (as in the unit dose forms of the invention) are bioequivalent to separate administration of the two APIs. An open-label, randomized, single-dose, oral administration, two-period crossover study was conducted. Six male subjects were assigned randomly to Sequence 1 or Sequence 2:

Sequence 1
Period 1: 800 mg ibuprofen [Motrin®], followed 24 hr later by 40 mg famotidine [Pepcid®].
Period 2: Concurrent administration of 800 mg ibuprofen and 40 mg famotidine.

Sequence 2
Period 1: Concurrent administration of 800 mg ibuprofen and 40 mg famotidine.
Period 2: 800 mg of ibuprofen, followed 24 hr later by 40 mg famotidine.

Following administration of ibuprofen and famotidine plasma ibuprofen and/or famotidine concentrations were determined in samples collected predose and at 0.25, 0.5, 1.0, 1.5, 2, 4, 6, 8, 10, 12, 14, 18, and 24 hr after administration of ibuprofen and/or famotidine. Ibuprofen and famotidine plasma concentrations, and computed pharmacokinetic parameters, were listed and summarized by dose (mean, standard deviation, 95% confidence interval, minimum, maximum). Individual and mean (by time) concentration-versus-time curves for each treatment, plotted on a semi-log scale, were examined. Intra-subject comparisons were made between Period 1 and Period 2.

WinNonLin version 2.1 was used to analyze the pharmacokinetic parameters from the concentration-versus-time data based a non-compartmental model. The pharmacokinetic values then were transferred to MS Excel or Graphpad Prism for calculation of means, SDs, confidence intervals, etc., for preparation of tables and figures, and for performance of statistical testing.

Analyses of variance appropriate for a two-period crossover design were performed on the computed parameters including terms for sequence, subject within sequence, formulation, and period. Analyses were performed on the observed data and on natural logarithm-transformed data for area under the concentration-versus-time curve (AUC) and maximum observed plasma concentration (Cₘₐₓ). Ninety-five (95) % confidence intervals were computed for the differences in treatment means.

After confirming the absence of a period effect for the pharmacokinetic parameters, individual AUC and Cₘₐₓ data were pooled for each treatment (i.e., for both ibuprofen and famotidine administered alone and in combination) for bioequivalence testing. The individual data then were log-transformed (natural log) and the differences for each drug between administration alone versus in combination were determined for each subject. The means and 95% confidence intervals of these log-transformed differences were calculated, and the upper and lower bound of the log-transformed range were normalized and then tested for bioequivalence. These intervals were evaluated in relation to the criterion equivalence interval of 80% to 125% for log-transformed data. Tables 1-3 show the results of the analyses:

TABLE 1
Pharmacokinetic Parameters (mean ± SD, 95% CI) for Ibuprofen and
Famotidine When Administered Alone and In Combination

| | Ibuprofen | | Famotidine | |
|---|---|---|---|---|
| Parameter | Alone | With Famotidine | Alone | With Ibuprofen |
| tₘₐₓ (hr) | 1.58 ± 0.49 | 2.25 ± 1.89 | 1.67 ± 0.52 | 2.17 ± 0.93 |
| (95% CI) | (1.07–2.10) | (0.27–4.23) | (1.13–2.21) | (1.19–3.14) |
| Cₘₐₓ | 56.279 ± 8.486 | 55.666 ± 12.106 | 143 ± 31 | 159 ± 50 |
| (ng/mL) | (47.374–65.184) | (42.961–68.370) | (111–175) | (107–211) |
| (95% CI) | | | | |
| t_{1/2} (hr) | 2.50 ± 0.55 | 2.56 ± 0.59 | 3.66 ± 0.19 | 3.49 ± 0.35 |
| (95% CI) | (1.92–3.07) | (1.95–3.18) | (3.46–3.86) | (3.12–3.85) |
| Kₑₗ | 0.29 ± 0.06 | 0.28 ± 0.06 | 0.19 ± 0.01 | 0.20 ± 0.02 |
| (95% CI) | (0.23–0.35) | (0.22–0.34) | (0.18–0.20) | (0.18–0.22) |
| AUC₍ₗₐₛₜ₎ | 236.992 ± 62.862 | 234.851 ± 67.655 | 883 ± 173 | 934 ± 275 |
| (ng/mL · hr) | (171.023–302.961) | (163.851–305.850) | (701–1064) | (646–1222) |
| (95% CI) | | | | |
| AUC | 245.124 ± 63.697 | 235.156 ± 67.749 | 893 ± 175 | 944 ± 279 |
| (ng/mL · hr) | (178.279–311.970) | (164.058–306.254) | (710–1077) | (651–1236) |
| (95% CI) | | | | |

TABLE 2
Bioequivalence Test Results for AUC (log-transformed values)
for Ibuprofen and Famotidine When Administered
Alone Versus In Combination

| | | AUC₍ₗₐₛₜ₎ | | |
|---|---|---|---|---|
| Drug | AUC(ₗₐₛₜ₎ Alone | In Combination | Difference | 95% CI |
| Ibuprofen | 12.35 | 12.33 | 0.02 | 0.94–1.11 |
| Famotidine | 6.765 | 6.799 | −0.034 | 0.79–1.19 |

¹Test criterion: CI within 0.8–1.25

TABLE 3
Bioequivalence Test Results for Cₘₐₓ (log-transformed values)
for Ibuprofen and Famotidine When Administered
Alone Versus In Combination

| | Cₘₐₓ | Cₘₐₓ | | |
|---|---|---|---|---|
| Drug | Alone | In Combination | Difference | 95% CI |
| Ibuprofen | 10.93 | 10.91 | 0.02 | 0.85–1.23 |
| Famotidine | 4.94 | 5.02 | −0.08 | 0.76–1.12 |

¹Test criterion: CI within 0.8–1.25

There were no significant differences between the treatment means for the pharmacokinetic parameters for either ibuprofen or famotidine when administered alone versus in combination. It was concluded that both ibuprofen and famotidine can be considered bioequivalent when administered in combination compared to separate administration.

### 14.4 Example 4

### Ibuprofen-Famotidine Compatibility Studies

As shown in Table 4, substantial degradation of famotidine was observed in the famotidine-ibuprofen mixture (1:29 ratio) under stress conditions in the presence of ibuprofen. In the absence of ibuprofen, famotidine is stable.

TABLE 4
Famotidine/Ibuprofen Stability Under Stress Conditions

| | | Famotidine |
|---|---|---|
| API | Storage condition | Content* |
| Famotidine | 2 weeks at 60° C. | 98% |
| Famotidine + Ibuprofen | 2 weeks at 60° C. | 81% |
| Famotidine + Ibuprofen | 1 mo at 40° C./75% RH | 54% |

*Famotidine content was determined by analytical HPLC and expressed as percent of target content.

Similarly, as shown in Table 5 substantial degradation of famotidine was observed in the tablet dosage form containing ibuprofen in a tablet formulation under stress conditions. The tablets contained 10 mg famotidine, 800 mg ibuprofen and the following excipients: pregelatinized starch (Starch 1500); hydroxypropyl cellulose; colloidal silicon dioxide; microcrystalline cellulose (Emcocel®50M and 90M); SMCC (ProSolv®50); SMCC (ProSolv®90); low substituted HPC (LH-11); croscarmellose; sodium; and magnesium stearate

TABLE 5
Stability of Famotidine in Tablet Under Stress Conditions

| Drugs in Tablet | | Famotidine |
|---|---|---|
| Formulation | Storage condition | Content* |
| Famotidine (13.3 mg) + Ibuprofen | Initial | 100% |
| (400 mg) | | |
| Famotidine (13.3 mg) + Ibuprofen | 1 week at 60° C. | 39% |
| (400 mg) | | |
| Famotidine (13.3 mg) + Ibuprofen | 1 month at | 83% |
| (400 mg) | 40° C./75% RH | |

*Famotidine content was determined by analytical HPLC and expressed as percent of target content.

### 14.5 Example 5

### Additional Ibuprofen-Famotidine Compatibility Studies

Approximately 0.5 g famotidine API was mixed with 14.5 g ibuprofen. After grinding, API mixture was stored in glass vials under the conditions indicated. As shown in Table 6, substantial degradation of famotidine was observed.

TABLE 6
Famotidine/Ibuprofen Stability Under Stress Conditions

| | Ibuprofen (% control) | | | Famotidine (% control) | | |
|---|---|---|---|---|---|---|
| API Mixture | 1 wk 40° C. | 1 wk 60° C. | 2 wks 60° C. | 1 wk 40° C. | 1 wk 60° C. | 2 wks 60° C. |
| Famotidine | | | | 96.1 | 121.0 | 100.1 |
| Famotidine-Ibuprofen | 104.7 | 99.9 | 96.4 | 94.4 | 85.7 | 46.0 |

### 14.6 Example 6

### Determination of Dissolution

One method for determination of the rate and extent of dissolution can be carried out using the methods described in the United States Pharmacopeia and National Formulary 29th Revision, under the following conditions:

| | |
|---|---|
| Dissolution Apparatus: | Apparatus II (Paddles) |
| Dissolution Medium: | 50.0 mM Potassium Phosphate Buffer, pH 7.2 |
| Dissolution Medium | 900 mL |
| Volume: | |
| Temperature in Vessel: | 37.0° C. ± 0.5° C. |
| Speed: | 50 RPM |
| Sampling Time: | 10 min., 20 min., 30 min., 45 min., 60 min., and |
| | infinity @ 250 rpm for 15 min. |
| Sampling Volume: | 1 mL |
| Sinker: | None |

When desired, the dissolution medium or other parameters may be varied. Typically a unit dose form is added to the vessel and dissolution is started. At specified times a portion (e.g., 2 ml) of medium is withdrawn and the amount of API in solution is determined using routine analytical methods (e.g., HPLC).

### 14.7 Example 7

### Manufacture of Ibuprofen/Famotidine Unit Dose Forms

This example describes how to make a particular ibuprofen/famotidine unit dose form.

### A. Producing the Ibuprofen Core

TABLE 6

| Item | Material | % w/w | mg/tablet | Function/Supplier |
|---|---|---|---|---|
| 1. | Ibuprofen USP | 64.00 | 800 | API/BASF |
| 2. | Lactose Monohydrate NF (80M) | 24.00 | 300 | Binder/Kerry |
| | | | | Biosci. |
| 3. | Colloidal Silicon Dioxide NF (Cab-O-Sil | 0.48 | 6 | Glidant/Cabot |
| | M5P) | | | |
| 4. | Croscarmellose Sodium NF Ac-di-Sol | 2.40 | 30 | Disintegrant/FMC |
| 5A. | Hypromellose USP, Methocel E-5 LV | 1.44 | 18 | Binder/Dow |
| | Premium (Intragranular in dry mix) | | | |
| 5B | Hypromellose USP, Methocel E-5 LV | 0.48 | 6 | Binder/Dow |
| | Premium (Intragranular as solution) | | | |
| 6. | Purified Water USP | — | q.s. | |
| 7. | Prosolv SMCC 90 (silicified | 3.76 | 47 | Binder/JRS |
| | microcrystalline cellulose) | | | |
| 8. | Croscarmellose Sodium NF | 2.40 | 30 | Disintegrant/FMC |
| | (Ac-di-Sol) | | | |
| 9. | Colloidal Silicon Dioxide NF | 0.32 | 4.0 | Glidant/Cabot |
| | (Cab-O-Sil M5P) | | | |
| 10. | Magnesium Stearate NF | 0.72 | 9.0 | Lubricant/Peter |
| | | | | Greven |
| | Core tablet weight | 100.0 | 1250 | |

Items 1-5A are sifted through Quadra Comil 16-mesh and mixed (Blend 1). Item 5B is dissolved in water and slowly added to Blend 1 using a mixer. Additional water is added and mixed. The wet material is dried at 50° C. for 12 h, milled using a 16-mesh screen with appropriate spacer, and dried until the LOD at 50° C. is below 0.5% w/w. Dried granules and extra granular material is transferred to a 3 cu. ft. V blender and mix for 3 minutes.

Items 7-9 are sifted through Quadra Comil using 16-mesh screen with appropriate spacer.

Item 10 (lubricant) is sifted through 30 mesh hand screen and transferred to the above blender and mixed for 3 minutes. The final blend is compressed into tablets using a DC 16 compression machine set with 0.3750×0.8125 caplet shaped punches. The target tablet weight is 1250 mg with range of 3.0% and hardness of 10-20 Kp.

### B. Barrier Layer

The compressed tablets are coated with Opadry II white (Y-22-7719) according to manufacturer's instructions to a weight gain of 1.5-2.0% w/w.

### C. Famotidine layer

Famotidine and Opadry II (Colorcon) are mixed at a 1:1 ratio and the unit dose form amount of famotidine is applied by spray coating.

### D. Over Coating Layer

Opadry II white is applied over the famotidine layer by spray coating.

All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention having now been described by way of written description and example, those of skill in the art will recognize that the invention can be practiced in a variety of embodiments and that the foregoing description and examples are for purposes of illustration and not limitation of the following claims.

This application is related to U.S. Provisional Application No. 60/700,481, filed Jul. 18, 2005, and to copending U.S. patent application Ser. No. 11/______, filed Jul. 18, 2006, entitled “Medicaments Containing Famotidine and Ibuprofen and Administration Of Same” [Attorney Docket No. 026057-000500], and to co-pending U.S. patent application Ser. No. 11/______, filed Jul. 18, 2006, entitled “Methods and Medicaments For Administration of Ibuprofen” [Attorney Docket No. 026057-000300] both of which are hereby incorporated by reference in their entirety and for all purposes.

1. An oral dosage form comprising:
first portion comprising a therapeutically effective amount of famotidine;
a second portion comprising a therapeutically effective amount of ibuprofen,
wherein the first portion completely surrounds the second portion; and
a barrier layer disposed between the first and second portions
wherein the oral dosage form contains ibuprofen and famotidine in an amount and at a ratio therapeutically effective for three times per day dosing.

2. The oral dosage form of claim 1 wherein in an aqueous environment the ibuprofen and famotidine are released into solution rapidly.

3. The oral dosage form of claim 1 wherein the oral dosage form comprises ibuprofen and famotidine in a ratio in the range of 29:1 to 32:1.

4. The oral dosage form of claim 3 wherein the oral dosage form comprises ibuprofen and famotidine in a ratio in the range of 30:1 to 31:1.

5. The oral dosage form of claim 1 wherein the oral dosage form comprises about 750 mg to 850 mg ibuprofen and about 24 mg to 28 mg famotidine.

6. The oral dosage form of claim 1 wherein the oral dosage form comprises about 375 mg to 425 mg ibuprofen and about 12 mg to 14 mg famotidine.

7. The oral dosage form of claim 1 that comprises
from 750 mg to 850 mg ibuprofen and 24 mg to 28 mg famotidine; or,
from 575 mg to 625 mg ibuprofen and 24 mg to 28 mg famotidine; or
from 375 mg to 425 mg ibuprofen and 12 mg to 14 mg famotidine; or,
from 175 mg to 225 mg ibuprofen and 6 mg to 7 mg famotidine.

8. The oral dosage form of claim 7 that comprises
about 800 mg ibuprofen and about 26.6 mg famotidine or
about 600 mg ibuprofen and about 26.6 mg famotidine or
about 400 mg ibuprofen and about 13.3 mg famotidine or
about 200 mg ibuprofen and about 6.6 mg famotidine.

9. The oral dosage form of claim 1 comprising 750 mg to 850 mg ibuprofen and 24 mg to 28 mg famotidine, wherein the ibuprofen and famotidine are present in a ratio of 29:1 to 32:1

10. The oral dosage form of claim 1 that is a tablet.

11. The oral dosage form of claim 10 wherein the barrier layer comprises Opadry.

12. The oral dosage form of claim 10 wherein the first portion (famotidine layer) comprises Opadry.

13. The oral dosage form of claim 1 wherein at least 75% of the famotidine and at least 75% of the ibuprofen in the dosage form are released within 15 minutes when measured in a Type II dissolution apparatus according to the U.S. Pharmacopoeia at 37° C. in 50 mM potassium phosphate buffer, pH 7.2 at 50 rotations per minute.

14. The oral dosage form of claim 1 wherein said second portion comprises ibuprofen, 20-30% (w/w) lactose monohydrate; 0.1 to 2% colloidal silicon dioxide; 3-7% croscarmellose sodium; 1-3% hydroxy propyl methyl cellulose; 2-6% silicified microcrystalline cellulose and 0.1-2% magnesium stearate.

15. An oral dosage form comprising:
a first portion comprising a therapeutically effective amount of famotidine;
a second portion comprising a therapeutically effective amount of ibuprofen, wherein the first portion completely surrounds the second portion; and
a barrier layer disposed between the first and second portions,
wherein in an aqueous environment the ibuprofen and famotidine are released into solution rapidly, and wherein the ibuprofen and famotidine are present in a range of 30:1 to 31:1.

16. An oral dosage form comprising:
a first portion comprising a therapeutically effective amount of famotidine;
a second portion comprising a therapeutically effective amount of ibuprofen, wherein the first portion completely surrounds the second portion; and
a barrier layer disposed between the first and second portions,
wherein in an aqueous environment the ibuprofen and famotidine are released into solution rapidly, and wherein the solid oral dosage form contains about 800 mg ibuprofen and about 26.6 mg famotidine or about 400 mg ibuprofen and about 13.3 mg famotidine.

17. An oral dosage form comprising:
a first portion comprising a therapeutically effective amount of famotidine;
a second portion comprising a therapeutically effective amount of ibuprofen, wherein the first portion completely surrounds the second portion; and
a barrier layer disposed between the first and second portions,
wherein in an aqueous environment the ibuprofen and famotidine are released into solution rapidly,
and wherein the solid oral dosage form is a tablet comprising an ibuprofen-containing core portion surrounded by a famotidine-containing layer and a barrier layer interposed between the core portion and famotidine-containing layer, and wherein the core portion comprises ibuprofen, 20-30% (w/w) lactose monohydrate; 0.1 to 2% colloidal silicon dioxide; 3-7% croscarmellose sodium; 1-3% hydroxy propyl methyl cellulose; 2-6% silicified microcrystalline cellulose and 0.1-2% magnesium stearate.

18. A method of treating a patient in need of ibuprofen treatment comprising administering one or more oral dosage forms according to claim 1, wherein the administering is on a three times per day (TID) schedule.

19. The method of claim 18 wherein the patient is at elevated risk for developing an NSAID-induced ulcer.

20. The method of claim 18 wherein said TID administration of said dosage form provides better gastric protection over a 24-hour period than TID administration of the same daily quantity of ibuprofen and two times a day (BID) administration of the same daily quantity of famotidine.

21. The method of claim 20 wherein the subject's intragastric pH is greater than 3.5 for at least 18 hours of a 24 hour dosing cycle.

22. The method of claim 18 wherein the patient's intragastric pH is greater than 3.5 for at least 20 hours of a 24 hour dosing cycle during a course of treatment with said oral dosage form.

23. The method of claim 18 wherein the subject is in need of ibuprofen treatment for a chronic condition.

24. The method of claim 23 wherein the chronic condition is rheumatoid arthritis, osteoarthritis or chronic pain.

25. The method of claim 18 wherein the subject is in need of ibuprofen treatment for acute pain, dysmenorrhea or acute inflammation.
